(19) United States
(12) Patent Application Publication
(10) Pub. No.: US 2022/0160338 A1
(43) Pub. Date: May 26, 2022

(54) FOLDABLE STOOL SPECIMEN COLLECTION DEVICE

(71) Applicant: University of South Alabama, Mobile, AL (US)

(72) Inventors: Elizabeth Christine Abts, Spanish Fort, AL (US); Arthur E. Frankel, Mobile, AL (US); Thomas Grant Glover, Spanish Fort, AL (US)

(73) Assignee: University of South Alabama, Mobile, AL (US)

(21) Appl. No.: 17/602,308

(22) PCT Filed: Apr. 8, 2020
(86) PCT No.: PCT/US2020/027133

§ 371 Date: Oct. 8, 2021

Related U.S. Application Data
(60) Provisional application No. 62/831,216, filed on Apr. 9, 2019.

(51) Int. Cl. A61B 10/00

(52) U.S. Cl. CPC A61B 10/0038

(57) ABSTRACT

A single-use foldable stool specimen collection device supportable atop the top face of a toilet seat is described. The device includes (a) a U-shaped foldable support member having a top surface, a bottom surface, an exterior U-shaped edge and an interior U-shaped edge defining a stool collection aperture; and (b) a foldable stool collection pocket attached to said foldable support member along its bottom surface, wherein the collection pocket spans and extends below the stool collection aperture. A folded stool specimen collection device and a stool specimen collecting system are also disclosed.

### RELATED APPLICATION INFORMATION

The present application claims priority to international application number PCT/US2020/027133 having a filing date of Apr. 8, 2020 and published as WO 2020/210273A1, which in turn claims priority to U.S. Provisional Application Ser. No. 62/831216 having a filing date of Apr. 9, 2019; both applications entitled “FOLDABLE STOOL SPECIMEN COLLECTION DEVICE”, the entire content and description of which are expressly incorporated herein by reference.

### FIELD OF THE INVENTION

The present invention generally pertains to a device for collecting solid human waste, for example feces or stool, upon elimination from a human body. More particularly, the present invention pertains to a foldable, single-use device for collecting stool upon elimination from the human body so that the stool specimen or a sample portion thereof may be analyzed.

### BACKGROUND OF THE INVENTION

The analysis of human feces or stool plays a critical role in many facets of today's medical practices. For example, a person's physician might request a stool culture to identify diseases of digestive tract, liver, and pancreas or the cause of an infection such as a virus, fungus or illness-causing bacteria such as shigella, salmonella, yersinia, campylobacter or E. coli. The presence of parasites, such as pinworms or Giardia can be detected by stool analysis. Enzymes such as trypsin or elastase may be evaluated in stool to help determine how well the pancreas is functioning. Fecal occult blood tests (FOBT) can detect minute amounts of blood in feces that cannot be seen with the naked eye and are therefore useful in diagnosing polyps and colorectal cancers. Newer assays using nucleic acid, protein or metabolite analysis are evaluated to detect cancer, infections, inflammation, and markers of diseases or response to therapies. Stool analysis can also detect poor absorption of nutrients by the digestive tract or even the previous consumption of illicit drugs.

As one might expect, careful collection and handling of stool to be used for such analytical and medical diagnostic purposes is essential for ensuring an accurate and reliable test result. Exposure of the stool to the external environment must be strictly minimized both in terms of time and the presence of potential sources for contamination such as contact with persons, other surfaces or materials and the air. Similarly, safety and proper hygiene demand both minimization of exposure for personnel who perform the stool collection and simple, fast disposal methods for any portion of the stool not retained for analysis.

Devices for stool collection that seek to achieve these requirements are known in the art but have various disadvantages. In one example, U.S. Published Patent Application Number 2012/0316462 describes a stool specimen collection system that includes a disposable bowl secured by a retainer to a frame which provides a rigid support for the bowl. The frame for the system, which is formed from material that is designed for repeated use and formed from materials designed for cycles of use, cleaning and sterilization, may be a source for sample and user contamination over time. Further, use of the system requires multiple user contact steps with the toilet seat (insofar that it is designed to be placed on a toilet bowl and under a toilet seat) which may be undesirable at best and unclean at worst. In another example, U.S. Pat. No. 5,463,782 describes a stool sample collection device including a sheet of preferably foldable material which has at least three extensions formed from the sheet which meet generally at an intersection and attachment apparatus such as a removable adhesive located at respective ends of the extensions distal from the intersection. This device could create additional risks of user exposure and specimen spillage during the application of pulling or twisting forces that may be required for removal necessitated by the attachment apparatus and, similar to '462 above, creates further undesirable interaction between the user and the toilet seat since the attachment apparatus attaches the device to the toilet bowl rim. Other devices such as the stool sample collector described in GB 2,558,687 and the foldable stool sample collection device described in EP 3,028,646 may be attached to a toilet seat; however, they require adhesive pads or the like for attachment to the seat which can introduce an additional contamination and spillage risk during the application of pulling or twisting forces required for removal. More generally, devices which employ adhesive attachment may, upon detachment, leave behind undesirable adhesive residue on the user-contacting surface of the toilet seat.

An ongoing need therefore exists for a single-use stool specimen collection device that (i) is easily and conveniently shippable and storable prior to use; (ii) minimizes exposure of the specimen, patient and users such as medical personnel to exposure and contamination during use and (iii) facilitates safe and easy disposal of the specimen and any infectious components it might contain after use.

### SUMMARY OF THE INVENTION

In a first aspect, the present invention relates to a single-use foldable stool specimen collection device supportable atop the top face of a toilet seat. The single-use foldable stool specimen collection device of the present invention includes (a) a U-shaped foldable support member having a top surface, a bottom surface, an exterior U-shaped edge and an interior U-shaped edge generally defining a stool collection aperture; and (b) a foldable stool collection pocket attached to said foldable support member along its bottom surface, wherein the collection pocket spans and extends below the stool collection aperture.

In a second aspect, the present invention relates to a folded stool specimen collection device. The folded device of the present invention includes (a) a U-shaped foldable support member that includes a top surface, a bottom surface, an exterior U-shaped edge and an interior U-shaped edge, wherein the support member is folded along fold lines that include a midfold fold line, a first side fold line and a second side fold line that are spaced along the length of the support member and extend radially from the exterior U-shaped edge to the interior U-shaped edge; and (b) a foldable stool collection pocket attached to the foldable support member along the bottom surface thereof, wherein the collection pocket is folded along fold lines that include a central lateral fold line; two side lateral fold lines each extending generally parallel to the central fold line and a longitudinal fold line intersecting and extending generally perpendicular to the central fold line.

In a third aspect, the present invention relates to a stool specimen collection system. The system of the present invention includes a) a toilet that includes a bowl with an interior cavity and a top rim and a hinged seat that includes a top face and which is pivotable between a first upright position and a second position resting on the rim; and (b) a stool specimen collection device supported atop the top face of the seat, the device including (i) a U-shaped foldable support member having a top surface, a bottom surface, an exterior U-shaped edge and an interior U-shaped edge generally defining a stool collection aperture; and (ii) a foldable stool collection pocket attached to the foldable support member along the bottom surface thereof which spans and extends below the stool collection aperture into the interior cavity of the toilet bowl.

In a fourth aspect, the present invention relates to a method for forming a folded stool specimen device. The method of the present invention includes the sequential steps of (a) providing a stool specimen collection device that includes (i) a U-shaped foldable support member having a top surface, a bottom surface, an exterior U-shaped edge, an interior U-shaped edge that defines a stool collection aperture, and fold lines that include a midfold fold line, a first side fold line and a second side fold line that are spaced along the length of the support member and extend radially from the exterior edge to the interior edge; and (ii) a foldable stool collection pocket attached to the foldable support member along the bottom surface thereof, with the stool collection pocket including fold lines that include a central lateral fold line; two side lateral fold lines each extending generally parallel to the central fold line; and a longitudinal fold line intersecting and extending generally perpendicular to the central fold line; (b) folding the stool specimen collection device along the mid-fold line of the support member; (c) folding the once-folded stool specimen collection device along the side lateral fold lines of the stool collection pocket; and (d) folding the twice-folded stool specimen collection device along the side fold lines of the support member.

Further aspects of the invention are as disclosed and claimed herein.

### BRIEF DESCRIPTION OF THE DRAWINGS

The invention will be described in further detail below, and with reference to the accompanying drawings, wherein

FIG. 1 is a perspective view of the single-use foldable stool specimen collection device of the present invention;

FIG. 2 is a perspective view of the stool specimen collection system of the present invention;

FIG. 3 is a top plan view of the single-use foldable stool specimen collection device of the present invention;

FIG. 4a is a top plan view depicting the first folding step of a method for forming the folded stool specimen collection device of the present invention from the foldable stool collection device of FIG. 3;

FIG. 4b is a top plan view depicting the second folding step of a method for forming the folded stool specimen collection device of the present invention from the foldable stool collection device of FIG. 3; and

FIG. 4c is a top plan view depicting the third folding step of a method for forming the folded stool specimen collection device of the present invention from the foldable stool collection device of FIG. 3.

### DETAILED DESCRIPTION

In the first aspect, the present invention is directed to a single-use, foldable stool specimen collection device that is supportable atop the top face of a toilet seat. Broadly, the device 5 includes a foldable curved support member 10 that is preferably U-shaped and a foldable stool collection pocket 30. As described in more detail below, in one or more embodiments the device is flushable and accordingly in one or more embodiments the curved support member 10 and the collection pocket 30 are both formed from a flushable material.

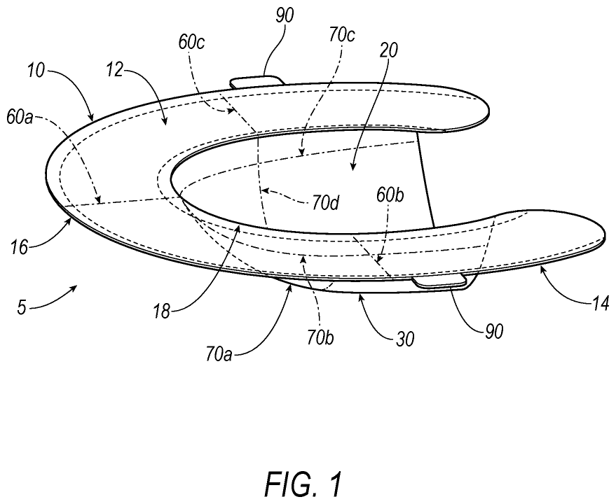

As depicted in FIGS. 1-3, the device 5 may include: (a) a U-shaped foldable support member 10 having a top surface 12, a bottom surface 14, an exterior U-shaped edge 16 and an interior U-shaped edge 18 that defines a stool collection aperture 20; and (b) a foldable stool collection pocket 30 attached to the foldable support member 10 along the bottom surface 14 thereof. The collection pocket 30 spans and extends below the stool collection aperture 20.

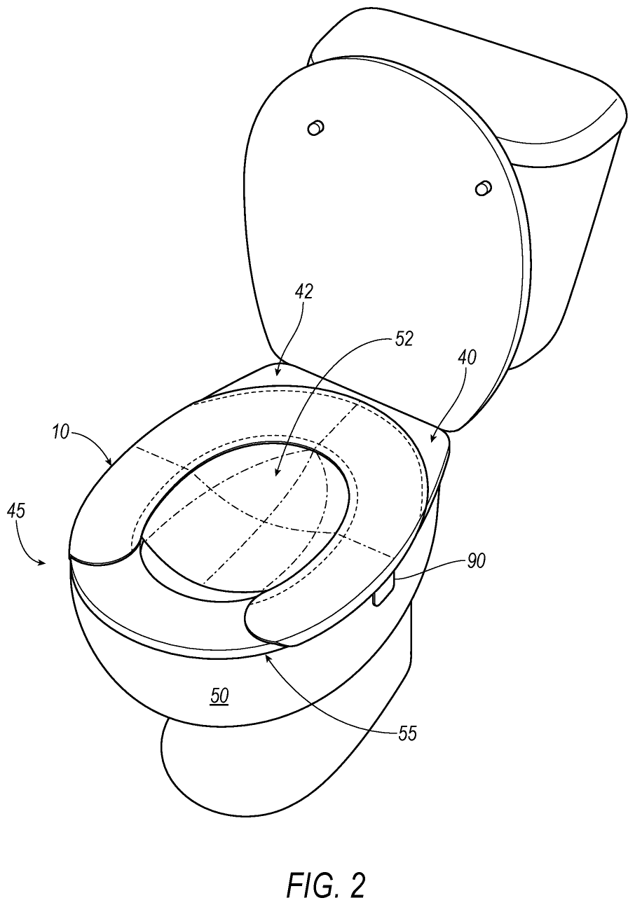

The device may be a component of a stool specimen collection system, another aspect of the present invention discussed in more detail below and depicted in FIG. 2. In use, the device 5 is supportable atop a conventional hinged toilet seat 40 of a conventional toilet 45 that includes a bowl 50 with an interior cavity 52 and a top rim 55. The hinged seat 40 includes a top face 42 and is pivotable between a first upright position and a second position resting on the top rim 55 of the bowl 50 as shown in FIG. 2. In one or embodiments, the bottom surface 14 the foldable support member 10 is supported atop the top face 42 of the toilet seat 40 and covers at least 30% or at least 40% or at least 50% or at least 60% or at least 70% or at least 80% or at least 90% of the total surface area of the top face 42 of the toilet seat 40 when said device 5 is in use. The foldable support member 10 may in some embodiments be described as a U-shaped support member and in some embodiments as a unitary support member. The term “unitary” connotes that it is the sole support member for supporting the device 5 atop the top face of the toilet seat 40. Nonetheless, one of ordinary skill will be appreciate that two or more foldable curved support members may be employed and may cover at least 30% or at least 40% or at least 50% or at least 60% or at least 70% or at least 80% or at least 90% of the total surface area of the top face 42 of the toilet seat 40. In such embodiments, the curved support members each include a curved interior edge and a curved exterior edge.

An important feature of the device of the present invention is that, in one or embodiments, the device is supportable atop the top face of a conventional toilet seat without additional purposeful seat attachment means. Additional purposeful seat attachment means are contemplated to be an element, feature or structure purposefully included as a component of the device to attach the device to the toilet seat. Non-limiting examples of additional purposeful attachment means include adhesives, adhesive pads or tabs, fasteners such as hook-and-loop fasteners, loops or the like. Accordingly, in one or more embodiments, the device is supportable atop the top face of a toilet seat in the absence of additional purposeful seat attachment means. In such embodiments, the device may be held in place atop the top face of the toilet seat by friction forces between the bottom surface of the foldable support member and the top face of the toilet seat and/or by the body weight of a person using the device, e.g. a person depositing a stool specimen into the device while sitting on the top surface 12 of the support member 10 while the device is supported atop the top face 42 of the toilet seat 40. In some specific embodiments, the bottom surface 14 of the support member 10 that is supportable atop the top face 42 of the toilet seat 40 is free of adhesive seat attachment means.

In one or more embodiments, the device of the present invention is foldable. Foldable is meant to generally connote that the device, and more particularly the curved support member 10 and collection pocket 30, are formed from materials with sufficient flexibility to bend and crease upon application of force thereto. In such embodiments, the device may include a plurality of fold lines. Though described as “lines”, one of ordinary skill will appreciate that the fold lines described herein are not necessarily linear or straight in form and may be for example curved such as with the lip fold lines described below. The support member 10 of the device 5 may include at least one and preferably a plurality of fold lines 60 along which the support member 10 may be folded. Fold lines 60 are preferably spaced along the length of the support member 10 and extend substantially across the width of the support member 10 radially from the exterior U-shaped edge 20 to the interior U-shaped edge 18. In one or more embodiments, the fold lines 60 may be solid or dotted visual indicators printed on or embossed onto the support member. Should the support member material be sufficiently stiff to make accurate folding along the fold lines difficult, the fold lines 60 may be solid or dotted tracks that are formed into the thickness of the material that makes up the support member 10 to increase the flexibility of the support member along the length of the tracks. Such fold lines may be formed for example by flexing the support member back and forth to form a crease or by stamping a pattern into the thickness of the support member to provide slight deformation or indentation thereto. In some embodiments, the fold lines 60 may be both visual indicators printed on the support member 10 and tracks formed into the support member 10. An exemplary embodiment is shown in FIGS. 1-3 in which the support member 10 includes a midfold fold line 60a, a first side fold line 60b and a second side fold line 60c, each extending substantially across the width of the support member 10 radially from the exterior U-shaped edge 20 to the interior U-shaped edge 18.

In one or more embodiments, the collection pocket 30 of the device 5 may also include fold lines 70 that may be one or both of tracks imprinted into the collection pocket and visual indicators printed on the collection pocket 30. In the exemplary embodiment shown in FIGS. 1-3, the collection pocket includes a central lateral fold line 70a; two side lateral fold lines 70b and 70c each extending generally parallel to the central fold line 70a; and a longitudinal fold line 70d intersecting and extending generally perpendicular to the central lateral fold line 70a and the side lateral fold lines 70b and 70c.

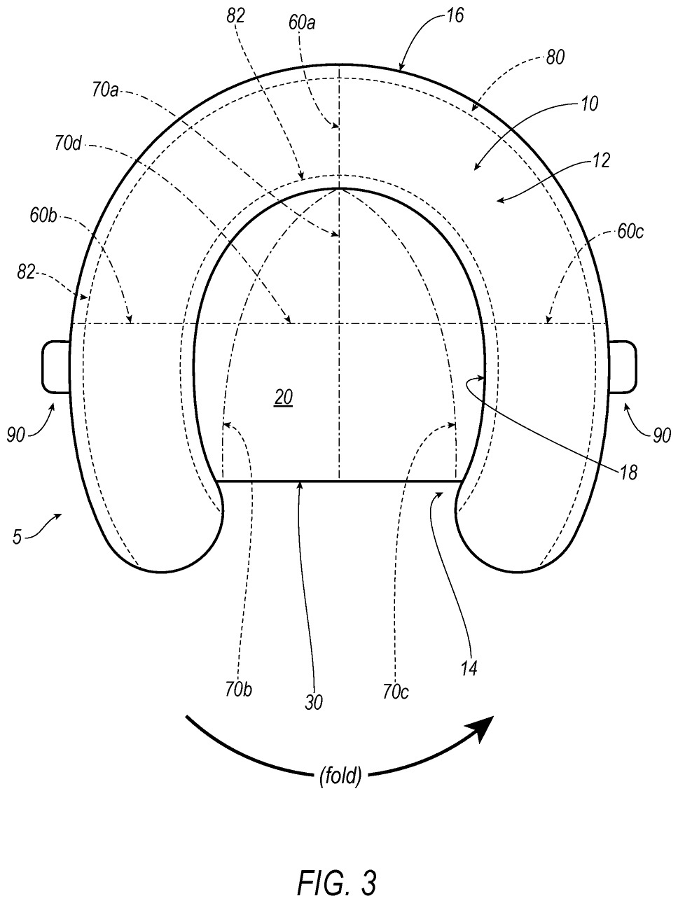

The device may include other additional features for enhancing ease of use and convenience. For example, the device 5 may include in one or more embodiments a lip 80 extending at least partially along at least one of the exterior edge 16 and the interior edge 18 of support member 10. In embodiments wherein the support member 10 is foldable, the lip 80 may be formable just prior to use of the device 5 by folding the support member 10 along one or more lip fold lines 82 printed on and/or formed in support member 10. Accordingly, the device of the present invention may include at least one lip fold line 82 extending at least partially along at least one of the exterior edge 16 and the interior edge 18 of the support member 10. A device 5 that includes lip fold lines 82 along both an exterior U-shaped edge 16 and an interior U-shaped edge 18 is depicted in FIG. 3 by way of example; however, it will be appreciated by one of ordinary skill that a plurality of lip fold lines 82 at each edge 16 and 18 may be provided so as to enhance usability of the device with toilet seats of various shapes and sizes.

In another example, the device 5 may include one or more grip tabs 90 extending radially outward from the exterior edge 16 of the support member 10. Grip tabs 90 may be grasped by a user after a stool specimen sample is deposited into the device to for example minimize shifting of the device as the user transitions from a sitting to standing position as well as to prevent inadvertent transference of the device to the user or away from or into the toilet bowl.

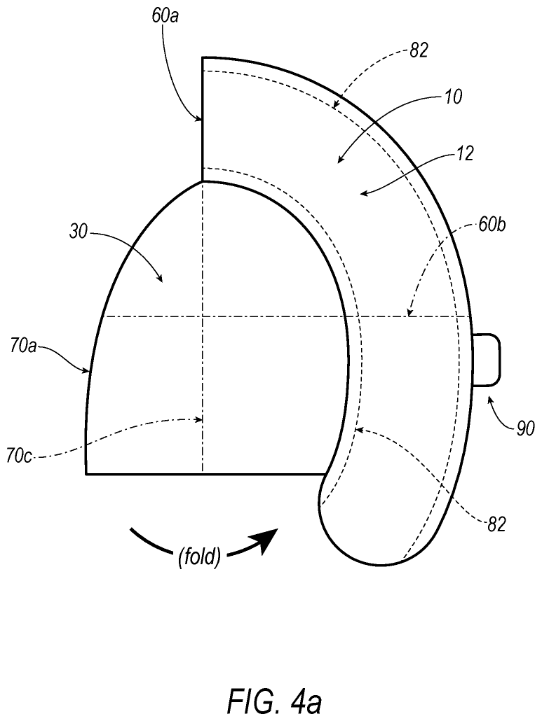

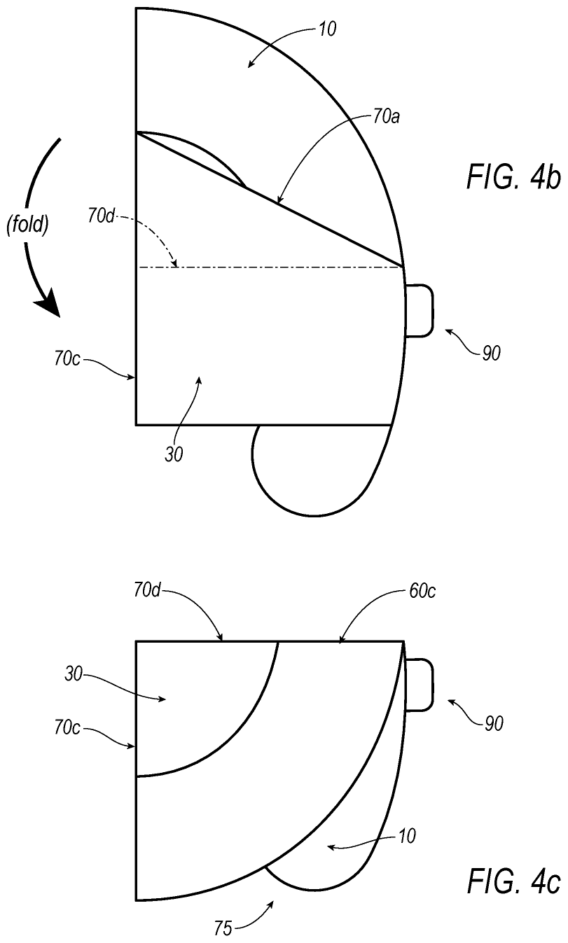

The fold lines 60 and 70 as shown in FIGS. 1-3 are intended to be illustrative of one embodiment of the folded stool specimen collection device of the present invention that is compact, readily shippable, easily storable and simply expandable from folded to unfolded form for use and which is described in more detail below and shown in FIGS. 4a through 4c. One of ordinary skill will appreciate, however, that other arrangements of fold lines may be contemplated and are within the scope of the present invention. Further, in one or embodiments, including embodiments in which the device may be foldable, the device of the present invention may be free of fold lines such that manufacturers of the device can choose the folding steps and methods most suitable for their specific devices and manufacturing and packaging preferences.

The stool collection pocket 30 is attached to the support member 10 along the bottom surface 14 of the support member 10. In one or more embodiments, stool connection collection is releasably attached to the support member, for example via use of a low-tack adhesive top the attach the pocket 30 to the support member 10 or by including perforations in the pocket 30.

In one or more embodiments, at least one of the support member 10 and the stool collection pocket 30 are flushable. Accordingly, the support member 10 is flushable in one or more embodiments. The stool collection pocket 30 is flushable in one or more embodiments. Preferably, both the support member 10 and stool collection pocket 30 are flushable. Most preferably, the device 5 of the present invention is flushable. The term “flushable”, as used herein to describe various aspects and elements of the present invention, is meant to mean that the aspect or element is generally dissolvable, disintegratable or dispersible in moving water (such as present in a sewage system) over time. More specifically, it may mean that it meets or exceeds one or more industry or government standards applicable to determining the disintegration in water of tissue paper and tissue products. Examples of such standards include proposed ISO 12625-17 and those promulgated by such organizations as the Association of the Nonwoven Fabric Industry or the International Water Services Flushability Group.

More generally, the device 5 of the present invention and its components may be constructed from any number of materials which balance a number of factors relevant to the use of the device in collecting stool specimens and more particularly in a stool specimen collection system as described below. Such factors include without limitation longitudinal/transversal pull strength and tear strength in both wet and dry conditions, stiffness, flexibility and foldability, surface friction characteristics and the like. Suitable materials may be films, fabrics, mesh, woven webs, nonwoven webs or laminates of various construction. Particularly suitable materials in one or more embodiments are flushable materials as that term is defined previously. Suitable flushable materials are described for example in U.S. Published Patent Application Nos. US2011/0270125A1 and US2018/0256136A1, the contents and disclosure of which are hereby incorporated herein by reference. One of ordinary skill will appreciate that the selection of materials for the device generally and the support member and collection pocket specifically should allow for and support functioning of the invention and its elements in the various aspects and embodiments as set forth herein. In one or more embodiments, the support member 10 and the stool collection pocket 30 are formed from separate pieces of flushable material having the same chemical composition with the separate pieces (and therefore the support member and the collection pocket) having differing relative thicknesses, densities or flexibilities. In one or embodiments, the foldable collection pocket 30 is more flexible, or thinner in cross-section, or less dense, than the foldable support member 10. In one or more embodiments, the foldable stool collection pocket 30 and the foldable support member 10 are formed from flushable materials of different chemical composition. In one or more embodiments, the support member and the stool collection pocket are formed from the same flushable material.

In another aspect, depicted in FIG. 4c and with reference to the remaining FIGs, the present invention is directed to a folded stool specimen collection device. In this aspect, the stool specimen collection device 75 is in a compact, folded form suitable for convenient packaging in an envelope or the like and shipping prior to use. In this aspect, the folded stool specimen collection device includes (a) a U-shaped support member including a top surface, a bottom surface, an exterior edge and an interior edge, wherein the support member is folded along fold lines spaced along the length of the support member and extending radially from its exterior edge to its interior edge; and (b) a stool collection pocket attached to the support member along its bottom surface and including fold lines, wherein the collection pocket is folded along the fold lines. In one or more embodiments, the device is folded along fold lines 60 and 70 as described herein and accordingly the folded stool specimen collection device 75 includes (a) a U-shaped foldable support member 10 that includes a top surface 12, a bottom surface 14, an exterior U-shaped edge 16 and an interior U-shaped edge 18, wherein the support member is folded along fold lines 60 that include a midfold fold line 60a, a first side fold line 60b and a second side fold line 60c that are spaced along the length of the support member and extend radially from the exterior edge 16 to the interior edge 18; and (b) a foldable flexible stool collection pocket 30 attached to the foldable support member 10 along the bottom surface 14 thereof, wherein the collection pocket 30 is folded along fold lines 70 that include a central lateral fold line 70a; two side lateral fold lines 70b and 70c each extending generally parallel to the central fold line 70a; and a longitudinal fold line 70d intersecting and extending generally perpendicular to the central lateral fold line and the side lateral fold lines. In one or more embodiments, the folded stool specimen collection device 75, or one or both of the support member 10 and the stool collection pocket 30 are flushable as defined elsewhere herein.

The folded stool specimen collection device 75 may be formed by folding the foldable stool specimen collection device along its fold lines. A particularly suitable method for forming the folded stool specimen device of the present invention includes the sequential steps of (a) providing a stool specimen collection device that includes (i) a U-shaped foldable support member 10 having a top surface 12, a bottom surface 14, an exterior U-shaped edge 16, an interior U-shaped edge 18 that defines a stool collection aperture 20, and fold lines 60 that include a midfold fold line 60a, a first side fold line 60b and a second side fold line 60c that are spaced along the length of the support member and extend radially from the exterior edge 16 to the interior edge 18; and (ii) a radial flexible stool collection pocket 30 attached to the foldable support member 10 along the bottom surface 14 thereof, with the stool collection pocket including fold lines 70 that include a central lateral fold line 70a; two side lateral fold lines 70b and 70c each extending generally parallel to the central fold line 70a; and a longitudinal fold line 70d intersecting and extending generally perpendicular to the central lateral fold line and the side lateral fold lines; (b) folding the stool specimen collection device along the mid-fold line of the support member as shown in FIG. 4a; (c) folding the once-folded stool specimen collection device along the side lateral fold lines of the stool collection pocket as shown in FIG. 4b; and (d) folding the twice-folded stool specimen along the side fold lines 60b and 60c of the support member as shown in FIG. 4c. In one or embodiments, step (b) of the method further includes folding the stool specimen collection device along the central fold line 70a of the stool collection pocket. In one or more embodiments, step (d) in the method further includes folding the device along the longitudinal fold line 70d of the stool collection pocket.

In another aspect, the present invention is directed to a stool specimen collection system that generally includes the foldable stool specimen collection device of the present invention supported atop a conventional toilet seat resting on a toilet bowl. Accordingly, the present invention in this aspect is a stool collection system that includes a) a toilet 45, said toilet including a bowl 50 with an interior cavity 52 and a top rim 55; and a hinged seat 40 that includes a top face and which is pivotable between a first upright position (not shown) and a second position (as shown in FIG. 2) resting on the rim; and (b) a single-use foldable stool specimen collection device 5 supported atop the top face 42 of the seat 40, the device including (i) a U-shaped foldable support member 10 having a top surface 12, a bottom surface 14, an exterior U-shaped edge 16 and an interior U-shaped edge 18 defining a stool collection aperture 20; and (ii) a foldable stool collection pocket 30 attached to the foldable support member 10 along the bottom surface 14 thereof. The collection pocket 30 spans and extends below the stool collection aperture 20 into cavity 52. In one or more embodiments, the bottom surface 14 of the support member 10 is supported atop the top face 42 of the toilet seat 40 and covers at least 30% or at least 40% or at least 50% or at least 60% or at least 70% or at least 80% or at least 90% of the total surface area thereof. In one or more embodiments, the support member 10 is a unitary support member with the term “unitary” defined as elsewhere described herein. In one or embodiments, at least one of the support member 10 and the stool collection pocket 30, preferably both the support member 10 and stool collection pocket 30, and most preferably the device 5, are flushable as that term is described herein. In one or more embodiments, the device is supported atop the top face 42 of the toilet seat 40 in the absence of purposeful additional seat attachment means such as adhesive seat attachment means.

In one or more embodiments, the stool specimen collection device and the stool specimen collection system of the present invention further includes a specimen portion sampler for collecting a portion of a stool specimen deposited in the stool collection pocket. The sampler may be in any form suitable for securing a portion of stool from the specimen for further analytical testing such as for example an elongated stick or cylindrical member, rod, wand, spoon-like device or the like. In one or more embodiments, portion sampler may be flushable and preferably is formed from a flushable material.

An important feature of the present invention is that it is useful for securing stool specimens from children as well as adults. Accordingly, the stool collection specimen system of the present invention may further include a toilet seat adapter atop top surface 12 of the support member 10. Toilet seat adapters, also referred to in the art as supplemental toilet seats or “potty seats”, are well known and commercially available in the art for facilitating a child's use of a standard adult-sized toilet and are described for example in U.S. Pat. No. 5,524,295A, the contents and disclosure of which are hereby expressly incorporated herein by reference.

That which is claimed is:

1. A single-use, foldable stool specimen collection device supportable atop the top face of a toilet seat, said device comprising:
(a) a U-shaped foldable support member having a top surface, a bottom surface, an exterior U-shaped edge and an interior U-shaped edge defining a stool collection aperture, said bottom surface supportable atop said top face of said toilet seat; and
(b) a foldable stool collection pocket attached to said foldable support member along said bottom surface, said collection pocket spanning and extending below said stool collection aperture.

2. The device of claim 1 wherein said foldable support member further comprises at least one fold line spaced along the length thereof and extending substantially across the width of said support member radially from said exterior U-shaped edge to said interior U-shaped edge.

3. The device of claim 2 wherein said flexible support member includes a midfold fold line, a first side fold line and a second side fold line each extending substantially across the width of said support member radially from said exterior U-shaped edge to said interior U-shaped edge.

4. The device of claim 3 wherein said stool collection pocket further comprises a central lateral fold line, two side lateral fold lines each extending generally parallel to said central fold line and a longitudinal fold line intersecting and extending generally perpendicular to said central fold line and said side lateral fold lines.

5. The device of claim 1 wherein at least one said flexible support member and said stool collection pocket are each formed from a flushable material.

6. The device of claim 1 wherein said flexible support member and said stool collection pocket are formed from a flushable material.

7. The device of claim 6 wherein said foldable collection pocket is more flexible, or thinner in cross-section, or less dense, then said foldable support member.

8. The device of claim 6 wherein said support member and said stool collection pocket are formed from different flushable materials.

9. The device of claim 1 wherein said device is flushable.

10. The device of claim 1 wherein said bottom surface of said support member is supportable atop said top face of said toilet seat to cover at least 30% of the total surface area of said top face of said toilet seat.

11. The device of claim 1 wherein said device is supportable atop the top face of a toilet seat in the absence of additional purposeful seat attachment means.

12. The device of claim 10 wherein said foldable support member is free of adhesive seat attachment means.

13. The device of claim 1 wherein said stool collection pocket is releasably connected to said support member.

14. The device of claim 1 wherein said U-shaped foldable support member is a unitary support member.

15. A folded stool collection device, said device comprising:
(a) a U-shaped support member including a top surface, a bottom surface, an exterior edge and an interior edge, wherein said support member is folded along fold lines spaced along the length of said support member and extending radially from said exterior edge to said interior edge; and
(b) a stool collection pocket attached to said support member along said bottom surface and comprising fold lines, wherein said collection pocket is folded along said fold lines.

16. The device of claim 12 where said fold lines of said support member comprise a midfold fold line, a first side fold line and a second side fold line, each extending substantially across the width of said support member radially from said exterior U-shaped exterior to said interior U-shaped edge.

17. A stool collection system comprising:
(a) a toilet, said toilet including a bowl with an interior cavity and a top rim; and
a hinged seat, said seat comprising a top face and being pivotable between a first position and a second position resting on said rim; and (b) a foldable stool collection device supported atop said top face of said seat in said second position, said device including (i) a U-shaped foldable support member having a top surface, a bottom surface and an interior U-shaped edge defining a stool collection aperture, said support member supported atop said top face of said toilet seat in the absence of additional purposeful attachment thereto; and (ii) a stool collection pocket fitting said stool collection aperture and connected to said support member, said collection pocket extending below said stool collection aperture into said interior portion of said bowl.

18. The system of claim 17 wherein said foldable stool collection device is flushable.

19. The device of claim 1 further comprising one or more grip tabs extending radially outward from said exterior U-shaped edge of said U-shaped support member.

20. The device of claim 1 further comprising at least one lip fold line extending at least partially along at least one of said exterior U-shaped edge and said interior U-shaped edge of said U-shaped support member.
